(19) United States
(12) Patent Application Publication
(10) Pub. No.: US 2024/0390886 A1
(43) Pub. Date: Nov. 28, 2024

(54) TIMED SYSTEM FOR RAPID ASSAY

(71) Applicant: Cubit Diagnostics, Inc., Chicago, IL (US)

(72) Inventors: Jacob Hirsch, Encinitas, CA (US); Robert Eisele, Carlsbad, CA (US); Samuel Hartman-Pickerill, Chicago, IL (US)

(73) Assignee: Cubit Diagnostics, Inc., Chicago, IL (US)

(21) Appl. No.: 18/693,777

(22) PCT Filed: Sep. 26, 2022
(86) PCT No.: PCT/US22/44740

§ 371 Date: Mar. 20, 2024

Related U.S. Application Data
(60) Provisional application No. 63/248,114, filed on Sep. 24, 2021.

(51) Int. Cl. B01L 3/00

(52) U.S. Cl. CPC B01L 3/502; B01L 2200/0647; B01L 2200/16; B01L 2300/042; B01L 2300/0672; B01L 2300/18; B01L 2400/0475; B01L 2400/0644; B01L 2400/065

(57) ABSTRACT

The timed system for rapid assay is a cartridge system capable of receiving a sample system and performing an assay of the enclosed sample. The cartridge includes a reaction chamber for conducting the assay, with access between the sample system, reaction chamber, and reagent canisters controlled by the motion of a valve. Motion of the valve is in turn controlled by a gearing subsystem that allows for timed opening of various fluent and fluid lines between the sample system, reaction chamber, and reagent canisters.

### CROSS-REFERENCE TO RELATED APPLICATIONS

This application is a national phase application under 35 U.S.C. § 371 of International Application No. PCT/US2020/044740, filed Sep. 26, 2022, which claims priority to U.S. Provisional Patent Application No. 63/248,114, filed Sep. 24, 2021, the contents of all of which are incorporated herein by reference in their entireties.

### BACKGROUND

The present disclosure is directed to a rapid assay system, specifically a system utilizing timed gearing mechanisms for sample purification followed by assay of a biological sample including but not limited to nucleic acid amplification, immunoassays and the like.

Sample purification and assay frequently require complicated lab setups and a skilled user performing the assay. Fully automated setups are used, but usually require a large non-mobile footprint and significant equipment investment. In recent years, “lab on a chip” mechanisms have been popularized, but can still be complicated to use, both in the selection of reagents and performance of test steps. The tests may also require significant time investment by the user. This can increase the analysis time and cost of large batches of assays, increase error rates due to contamination or incorrect methodology, and limit mobility for field tests.

There is an unmet need in the art for an easy-to-use system for accurate biological assay requiring low time and equipment investment by a user.

### BRIEF SUMMARY

One embodiment of the present invention is a system for assay of a biological sample. The system includes a cartridge system. The cartridge system includes a cartridge frame to which is mounted a sample receiver connected to or configured to connect to a sample receptacle, at least one reagent receiver, at least one valve, and at least one reaction chamber; a plurality of fluid channels extending between the at least one valve, the sample receiver, the at least one reagent receiver, and the at least one reaction chamber; at least one fluent channel extending from a pressurization mechanism to the at least one valve; and a main drive gear, wherein rotation of the main drive gear determines the position of the at least one valve, wherein the position of the at least one valve determines which of the plurality of fluid channels is operably connected to the fluent channel. The pressurization mechanism can operate by negative, positive pressure, or a combination of both.

Another embodiment of the present invention is a system wherein the sample receiver comprises a sample heater operably connected to a system controller.

Another embodiment of the present invention is a system wherein the pressurization mechanism comprises a fixedly mounted syringe housing, and a syringe barrel slidably disposed within the syringe housing, wherein the fluent channel extends from the syringe housing to the at least one valve.

Another embodiment of the present invention is a system wherein the main drive gear is connected to a cam drive gear driving a rotating cam.

Another embodiment of the present invention is a system wherein the syringe barrel is connected to a syringe follower, the syringe follower being in contact with the cam.

Another embodiment of the present invention is a system wherein the syringe barrel is connected to a syringe spring, the syringe spring biasing the syringe barrel relative to the syringe housing.

Another embodiment of the present invention is a system wherein the main drive gear is a worm gear, and the cam drive gear comprises at least one cam gear ratchet and at least one cam gear pawl, wherein the combination of the at least one cam gear ratchet and at least one cam gear pawl forms an intermittent gear by allowing rotation of the cam drive gear in only one direction.

Another embodiment of the present invention is a system wherein the cartridge frame comprises a power source operably connected to a system controller.

Another embodiment of the present invention is a system wherein the system controller comprises a timer.

Another embodiment of the present invention is a system wherein the system controller comprises a gear actuator controlled by the timer, the gear actuator driving the main drive gear.

Another embodiment of the present invention is a system wherein the cartridge frame further comprises a readout.

Another embodiment of the present invention is a system wherein the at least one reaction chamber comprises a mixing mechanism operably connected to a system controller.

Another embodiment of the present invention is a system wherein the mixing mechanism comprises at least one vibrating actuator connected to at least one flexible membrane, wherein the at least one flexible membrane forms at least one wall of the at least one reaction chamber.

Another embodiment of the present invention is a system wherein the at least one reaction chamber comprises a sample heater operably connected to a system controller.

Another embodiment of the present invention is a system wherein the at least one reaction chamber comprises a magnet.

Another embodiment of the present invention is a system wherein the at least one reaction chamber is located within the at least one valve.

Another embodiment of the present invention is a system wherein the main drive gear is connected to at least one valve drive gear driving the at least one valve.

Another embodiment of the present invention is a system wherein a combination of the at least one valve drive gear and at least one valve gear forms an intermittent gear through incomplete toothing of the at least one valve drive gear.

Another embodiment of the present invention is a system wherein the intermittent gear is an intermittent rack-and-pinion gear, and the at least one valve is a sliding valve.

Another embodiment of the present invention is a system wherein the main drive gear is a worm gear, and the at least one valve drive gear comprises at least one valve gear ratchet and at least one valve gear pawl, wherein the combination of the at least one valve gear ratchet and at least one valve gear pawl forms an intermittent gear by allowing rotation of the at least one valve drive gear in only one direction.

Another embodiment of the present invention is a system wherein the at least one valve is a rotating valve.

Another embodiment of the present invention is a system further comprising a fluent flow within the fluent channel, wherein the fluent flow is isolated from ambient atmosphere.

Another embodiment of the present invention is a system wherein the cartridge frame comprises a first cartridge mounting element and a second cartridge mounting element separable from the first cartridge mounting element, the first cartridge mounting element includes the main drive gear mounted thereto, and the second cartridge mounting element includes the sample receiver, the at least one reagent receiver, and the at least one valve mounted thereto.

Another embodiment of the present invention is a system further comprising a sample system, the sample system comprising: a sample receptacle receiving the biological sample; and a sample cap, wherein a sample reservoir is located within the sample cap.

Another embodiment of the present invention is a system wherein a sample holder is connected to the sample cap.

Another embodiment of the present invention is a system wherein the sample receptacle further comprises at least one reservoir piercer.

Another embodiment of the present invention is a system of wherein the at least one reservoir piercer is placed within the sample receptacle such that the sample reservoir is pierced by the at least one reservoir piercer when the sample cap is connected to the sample receptacle.

Another embodiment of the present invention is a system wherein the sample receiver comprises a sample heater operably connected to a system controller; wherein the pressurization mechanism comprises a fixedly mounted syringe housing, and a syringe barrel slidably disposed within the syringe housing, wherein the fluent channel extends from the syringe housing to the at least one valve; wherein the main drive gear is connected to a cam drive gear driving a rotating cam; wherein the syringe barrel is connected to a syringe follower, the syringe follower being in contact with the cam; wherein the syringe barrel is connected to a syringe spring, the syringe spring biasing the syringe barrel relative to the syringe housing; wherein, optionally, the cartridge frame comprises a power source operably connected to a system controller; wherein the system controller comprises a timer; wherein the system controller comprises a gear actuator controlled by the timer, the gear actuator driving the main drive gear; wherein the cartridge frame further comprises a readout; wherein the at least one reaction chamber comprises a mixing mechanism operably connected to the system controller; wherein, optionally, the mixing mechanism comprises at least one vibrating actuator connected to at least one flexible membrane, wherein, optionally, the at least one flexible membrane forms at least one wall of the at least one reaction chamber; wherein the at least one reaction chamber comprises a sample heater operably connected to the system controller; wherein, optionally, the at least one reaction chamber comprises a magnet; wherein the main drive gear is connected to at least one valve drive gear driving the at least one valve; wherein a combination of the at least one valve drive gear and at least one valve gear forms an intermittent gear through incomplete toothing of the at least one valve drive gear, wherein the intermittent gear is an intermittent rack-and-pinion gear, and the at least one valve is a sliding valve, or wherein the main drive gear is a worm gear, the cam drive gear comprises at least one cam gear ratchet and at least one cam gear pawl, the combination of the at least one cam gear ratchet and at least one cam gear pawl forms an intermittent gear by allowing rotation of the cam drive gear in only one direction, the at least one valve drive gear comprises at least one valve gear ratchet and at least one valve gear pawl, the combination of the at least one valve gear ratchet and at least one valve gear pawl forms an intermittent gear by allowing rotation of the at least one valve drive gear in only one direction, and the at least one valve is a rotating valve; wherein, optionally, the system further comprises a sample system comprising the sample receptacle receiving the biological sample and a sample cap, wherein, optionally, a sample reservoir is located within the sample cap; wherein, optionally, a sample holder is connected to the sample cap; wherein, optionally, the sample receptacle further comprises at least one reservoir piercer; wherein, optionally, the at least one reservoir piercer is placed within the sample receptacle such that the sample reservoir is pierced by the at least one reservoir piercer when the sample cap is connected to the sample receptacle.

Another embodiment of the present invention is a method of using the system of any prior claim. The method comprises complexing a target nucleic acid in a first buffer in the sample receptacle with a solid substrate to generate a target nucleic acid complex in the first buffer, transferring the first buffer comprising the target nucleic acid complex from the sample receptacle to the at least one reaction chamber, immobilizing the target nucleic acid complex in the at least one reaction chamber, removing the first buffer from the at least one reaction chamber and the target nucleic acid complex immobilized therein, transferring an amplification reaction mix to the at least one reaction chamber, and amplifying the target nucleic acid in the amplification reaction mix in the at least one reaction chamber to generate amplified nucleic acid.

Another embodiment of the present invention is a method further comprising transferring the first buffer from a first reagent receiver to the sample receptacle.

Another embodiment of the present invention is a method wherein the first buffer, prior to complexing with the target nucleic acid, comprises the solid substrate.

Another embodiment of the present invention is a method wherein the first buffer, prior to complexing with the target nucleic acid, comprises a lysis reagent.

Another embodiment of the present invention is a method further comprising heating the first buffer and the target nucleic acid in the sample receptacle.

Another embodiment of the present invention is a method wherein the immobilizing the target nucleic acid complex in the at least one reaction chamber comprises immobilizing the target nucleic acid complex in the at least one reaction chamber with a magnet or immobilizing the target nucleic acid complex in a porous substrate contained within the at least one reaction chamber.

Another embodiment of the present invention is a method further comprising, before removing the first buffer from the at least one reaction chamber and prior to transferring the amplification reaction mix to the at least one reaction chamber, washing the immobilized target nucleic complex in the at least one reaction chamber, wherein the washing comprises transferring a wash solution from a second reagent receiver to and through the at least one reaction chamber.

Another embodiment of the present invention is a method wherein the transferring the amplification reaction mix to the at least one reaction chamber comprises transferring the amplification reaction mix from a third reagent receiver to the at least one reaction chamber.

Another embodiment of the present invention is a method further comprising, after the amplifying the target nucleic acid, transferring the reaction mix comprising the amplified nucleic acid to a readout, wherein the amplified nucleic acid is detected with the readout.

### BRIEF DESCRIPTION OF THE DRAWINGS

FIGS. 1a and 1b show exploded and cross-sectional views of an exemplary embodiment of a sample system.

FIGS. 1c, 1d, and 1e show perspective, cross-sectional, and cross-sectional in-system views of another exemplary embodiment of a sample system.

FIGS. 2a, 2b, 2c, and 2d show side, top, internal bottom, and bottom views of an exemplary embodiment of a cartridge system in combination with the exemplary embodiment of the sample system.

FIGS. 2e and 2f show top and bottom views of another exemplary embodiment of a cartridge system.

FIGS. 3a and 3b show exploded and perspective views of the exemplary embodiment of the sample system in combination with the exemplary embodiment of the cartridge system.

FIG. 4 shows a side view of the respective gearing rotations of an exemplary embodiment of the cartridge system.

FIGS. 5a, 5b, and 5c show perspective, cross-sectional, and cross-sectional in-system views of an exemplary embodiment of the reagent canister.

FIGS. 6a, 6b, 6c, 6d, and 6e show side views of flow diagrams for the exemplary embodiment of the cartridge system of FIGS. 2a-2d during progressive steps of an assay.

FIGS. 7a, 7b, 7c, 7d, 7e, and 7f show top, partial top, partial perspective, partial top, partial side, and partial side views of an exemplary embodiment of an at least partially reusable cartridge system.

The objects and advantages of the invention will appear more fully from the following detailed description of the embodiments of the invention.

### DETAILED DESCRIPTION OF THE INVENTION

In the present description, certain terms have been used for brevity, clarity and understanding. No unnecessary limitations are to be implied therefrom beyond the requirement of the prior art because such terms are used for descriptive purposes only and are intended to be broadly construed. The different compositions and methods described herein may be used alone or in combination with other compositions and methods. Various equivalents, alternatives and modifications are possible within the scope of the appended claims. Each limitation in the appended claims is intended to invoke interpretation under 35 U.S.C. § 112, sixth paragraph, only if the terms “means for” or “step for” are explicitly recited in the respective limitation.

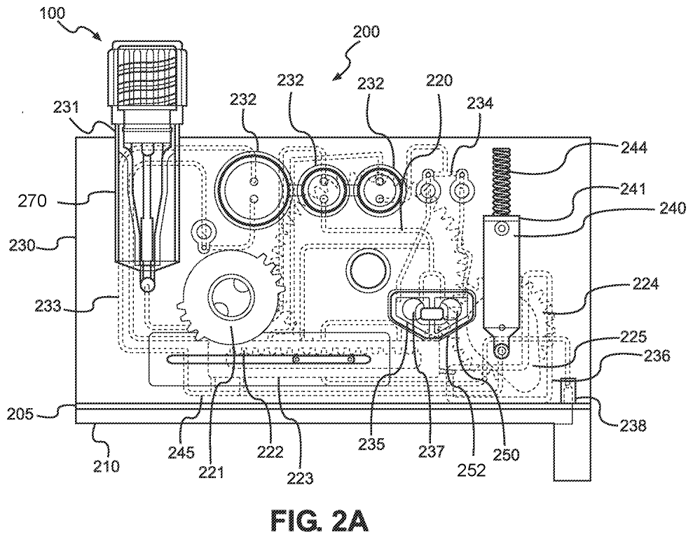

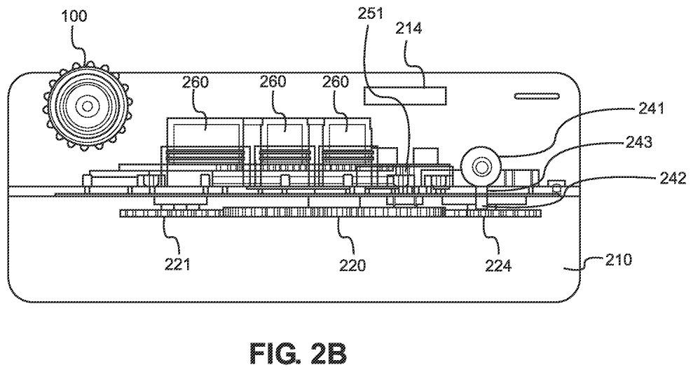

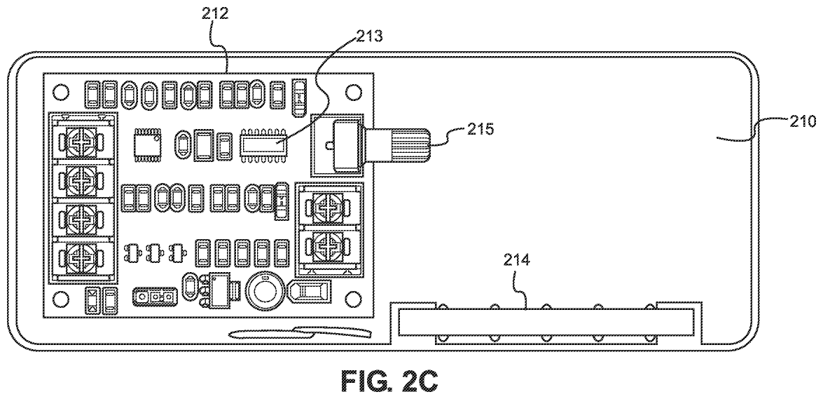

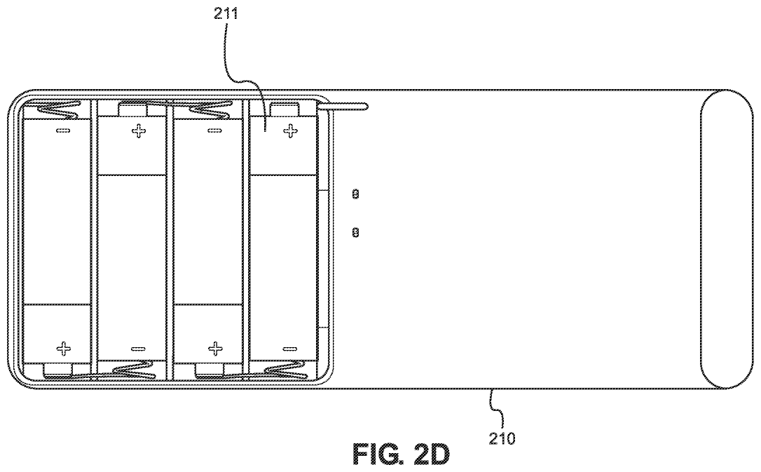

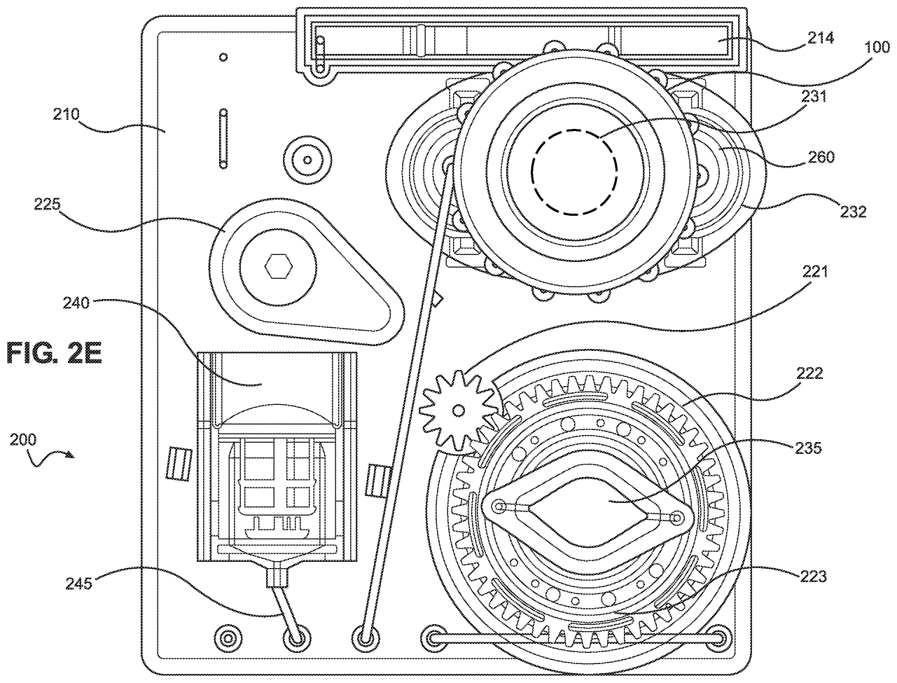

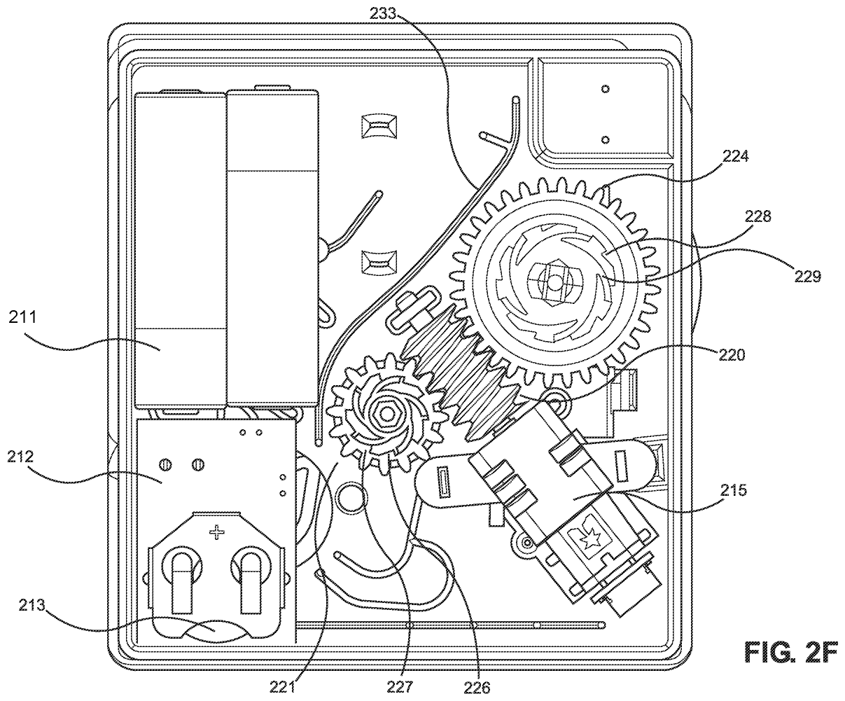

As shown in FIG. 2a, the assay system comprises at least two parts, a sample system 100 and a cartridge system 200.

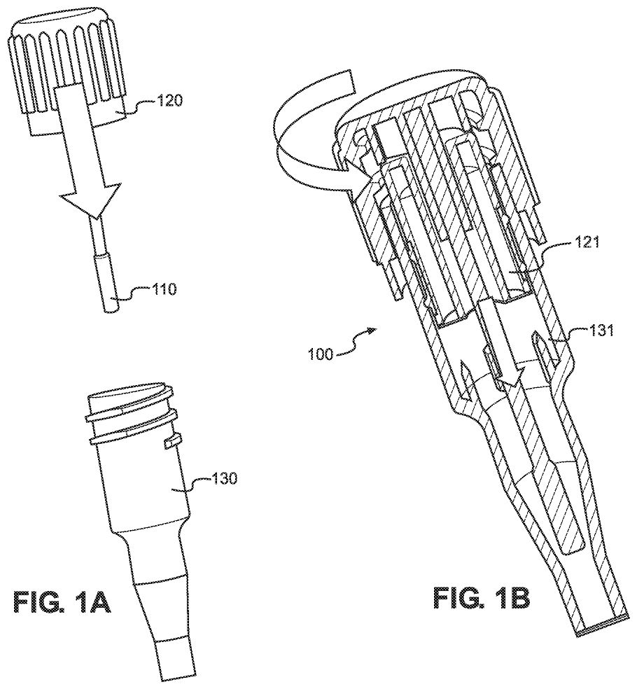

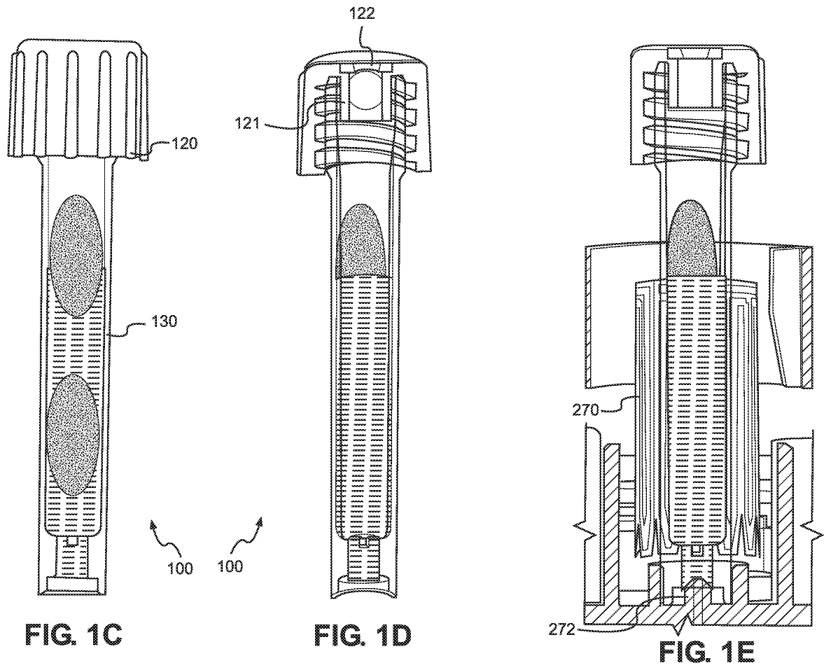

In certain embodiments, as shown in FIGS. 1a and 1b, upon sampling the biological matter to be tested, a sample holder 110 is inserted into a sample receptacle 130, which may include a sample buffer or at least one reagent. In some embodiments, the sample holder 110 comes pre-assembled to a sample cap 120. In some embodiments, the sample cap 120 can also include a closed sample reservoir 121 as an internal component. The reservoir 121 may contain a fluid or lyophilized consumable, such as, but not limited to, a sample buffer or prepared reagent or reagents for combination with the sample. This sample system 100 provides for consumable stability during storage via means such as foil laminate packaging. The holder 110 is inserted into a sample receptacle 130. Upon insertion, at least one reservoir piercer 131 punctures or cuts the reservoir 121, allowing the consumable to combine with the material in the sample reservoir 121. The cap 120 may be held in place on the receptacle 130 by means of a threaded, snap, bayonet, or other connection. A user may need to perform inversions or other mixing steps to combine the consumable with the sample before inserting the sample system 100 into a cartridge system 200.

In certain other embodiments, as shown in FIGS. 1c and 1d, upon sampling the biological matter to be tested, the sample is inserted into a sample receptacle 130, which may include a sample buffer or at least one reagent, by a sample holder 110 which does not remain in the sample system, such as a pipette or swab. In some embodiments, the sample cap 120 can also include a closed sample reservoir 121 as an internal component. The reservoir 121 may contain a fluid or lyophilized consumable, such as, but not limited to, a sample buffer or prepared reagent or reagents for combination with the sample. This sample system 100 provides for consumable stability during storage via hermetic sealing means such as foil laminate packaging over sample cap 120 and/or sample receptacle 130. A pneumatic spike 271 may be inserted through a sample aperture 122 in the sample cap 120 to pressurize the sample receptacle 130. Upon insertion, the pneumatic spike 271 punctures or cuts open the reservoir 121 and forms a pneumatic seal with the sample aperture 122, allowing the consumable within to combine with the material in the sample reservoir 121. Once combined, a fluidic spike 272 may open the sample receptacle 130 opposite the sample cap 120 to allow egress to the cartridge system 200.

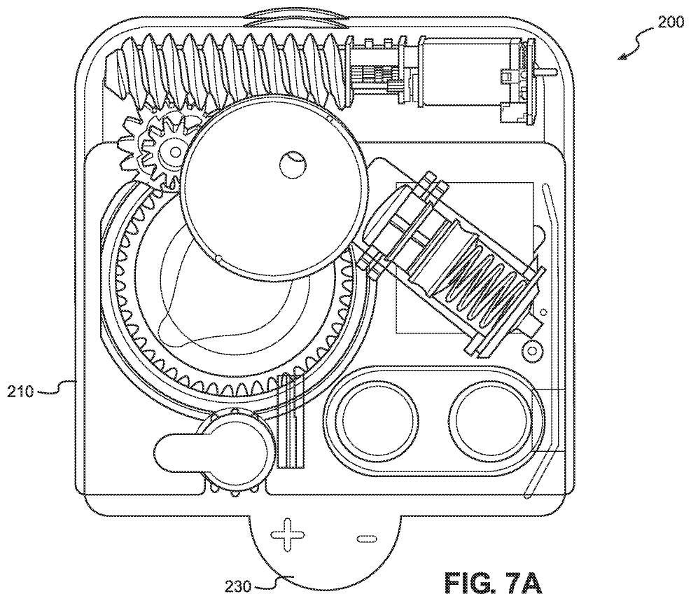

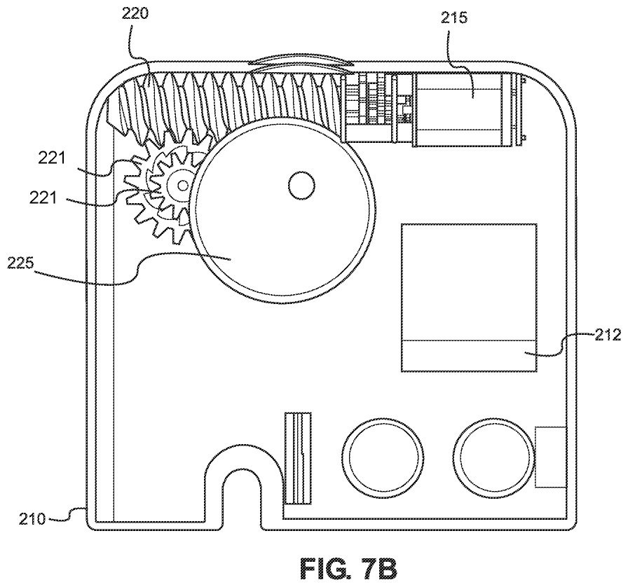

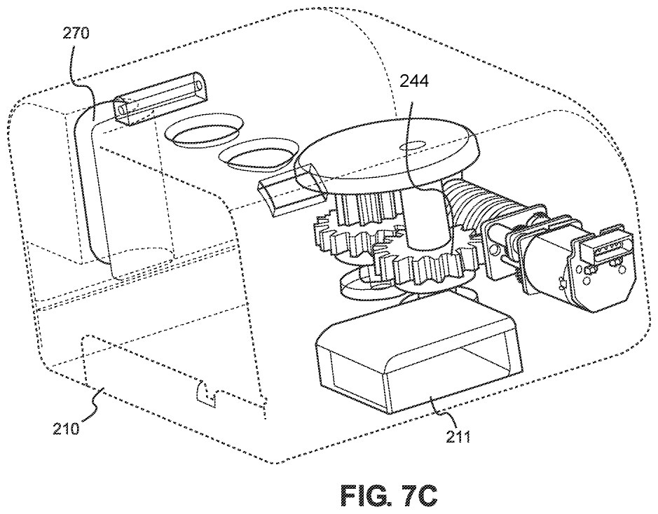

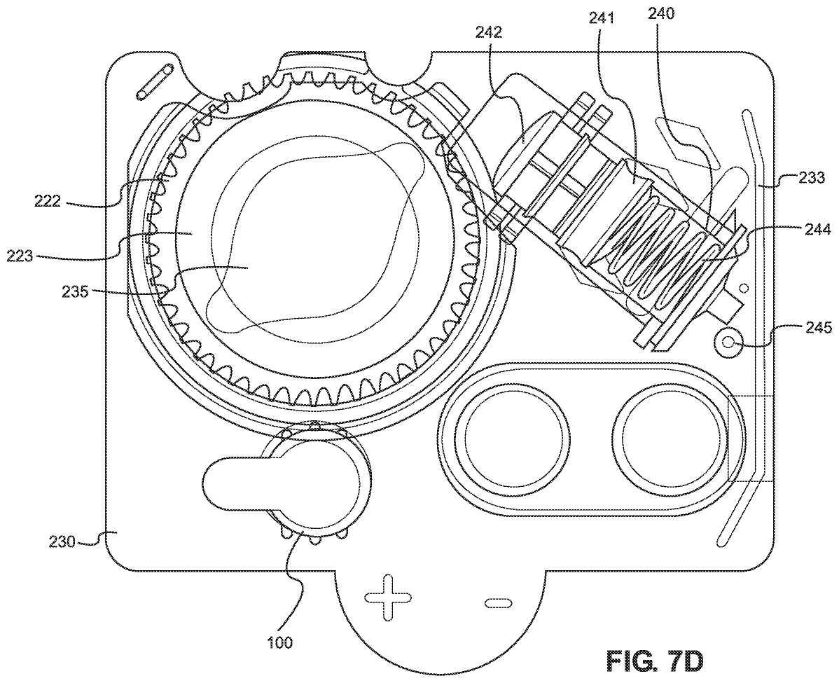

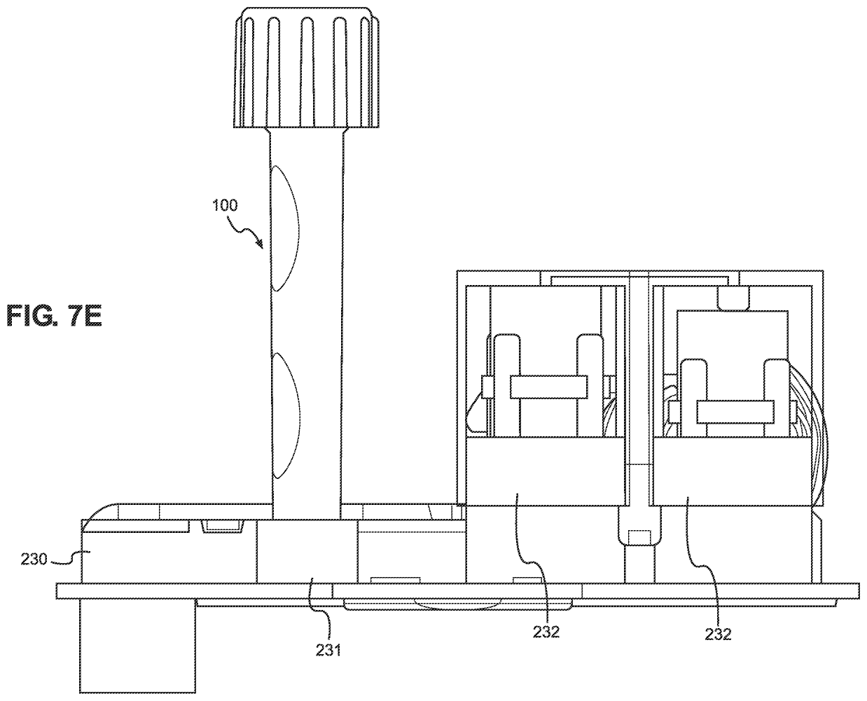

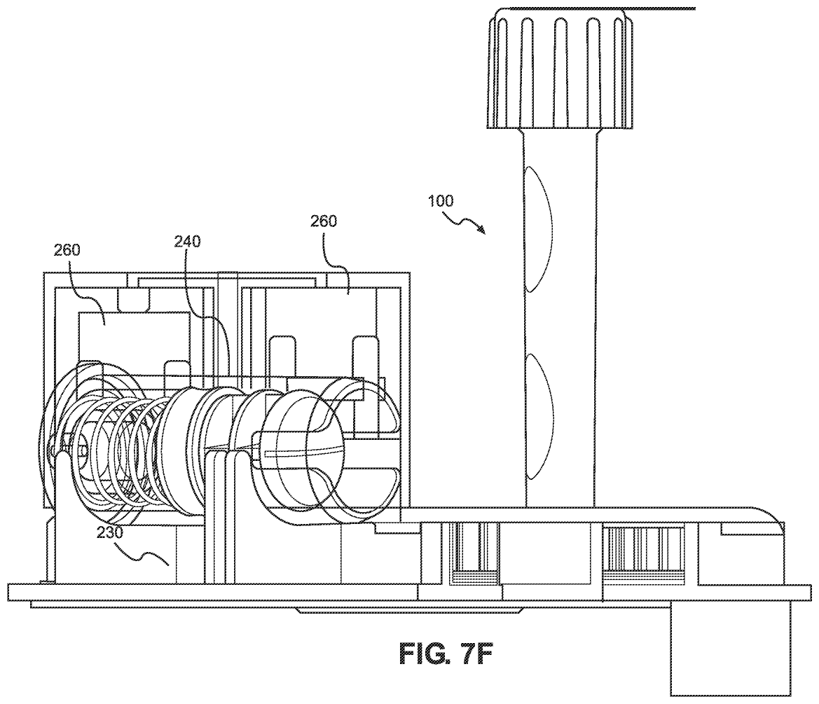

The cartridge system 200 includes a cartridge frame 205 to which are mounted other elements of the cartridge system 200. The cartridge frame 205 may be formed integrally or from a combination of separate cartridge mounting elements, which may or may not be separable by a user, depending on the embodiment. In certain embodiments, the cartridge frame 205 comprises at least one molded polymer cartridge mounting element. As shown in the embodiment of FIGS. 2a through 2d, the cartridge frame 205 may include a first cartridge mounting element 210 in the form of a base supporting a second cartridge mounting element 230 in the form of a wall extending therefrom. In the embodiment shown in FIGS. 2e and 2f, the cartridge frame 205 includes a first cartridge mounting element 210. In the embodiment shown in FIGS. 7a through 7f, the cartridge frame 205 comprises a first cartridge mounting element 210 surrounding a second cartridge mounting element 230. The second cartridge mounting element 230 in the embodiment shown in FIGS. 7a through 7f is insertable in the first cartridge mounting element 210 in a nested configuration and is also removeable therefrom, thereby permitting the first cartridge mounting element 210 to be reusable and the second cartridge mounting element 230 to be disposable.

As shown in FIGS. 2a through 2f, the first cartridge mounting element 210 includes a power source 211 electrically connected to a system controller 212. The power source 211 may be at least one battery cell. The system controller 212 may be at least one printed circuit board. The system controller 212 may include at least one timer 213 for controlling timing of the assay steps. At least one readout 214 may be included in the first cartridge mounting element, and may be an electronic readout or visual readout. In certain embodiments, the visual readout 214 is a lateral flow test pad housed on the first cartridge mounting element 210. The readout 214 may also be operably connected to sensors within the cartridge system 200 to enable readout via Near Field Communication (NFC) or Bluetooth Low Energy (BLE). The readout 214 can comprise any device or system capable of detecting amplified nucleic acid and displaying the detection to a user. A number of such devices or systems are known in the art. Exemplary readouts 214 include visual flow strips, fluorescent detection systems, etc.

The system controller 212 and timer 213 may be connected to at least one of a heating, motive, or mixing mechanism. In certain embodiments, the timer 213 is connected to a gear actuator 215, which is used to drive a main drive gear 220, thereby controlling the steps of the assay. In certain embodiments, the gear actuator 215 is a motor, such as, but not limited to, an electrical motor, while the timer 213 is an electronic timer. In other embodiments the gear actuator 215 is a drive spring such as, but not limited to, a pre-wound torsion spring that also provides mechanical timing. In other embodiments, a combination of electronic and mechanical timers is used to control different sub-systems.

The main drive gear 220 is interconnected with at least one valve drive gear 221 and at least one cam drive gear 224, both of which are mounted to the second cartridge mounting element 230 or first cartridge mounting element 210. The main drive gear 220, valve drive gear 221, and cam drive gear 224 may be spur gears, as shown in FIG. 2a. The main drive gear 220 may also be a worm gear, as shown in FIG. 2f. Further to the embodiment of FIG. 2f, valve drive gear 221, and cam drive gear 224 may be ratcheting gears, with the valve drive gear 221 having a valve gear ratchet 226 and a valve gear pawl 227, and the cam drive gear 224 having a cam gear ratchet 228 and a cam gear pawl 229. In an embodiment, the valve and cam gear pawls 227 and 229 are molded pieces with flexible fingers that give when in sliding contact with the ramping portions of the valve and cam gear ratchets 226 and 228, preventing the respective gear from being driven when the main drive gear 220 rotates in the incorrect direction. This allows the main drive gear 220 to drive only the valve drive gear 221 when rotated in a first direction and then drive only the cam drive gear 224 when rotated in a second direction. This embodiment may also be used when the main drive gear 220 is a spur gear.

As shown in FIGS. 2a through 2f, the valve drive gear 221 is interconnected with at least one valve gear 222 such that rotation of the valve drive gear 221 intermittently moves a valve 223. In certain embodiments, intermittent motion is due to incomplete toothing of the valve drive gear 221. In other embodiments, intermittent motion is due to intermittent and/or varying clockwise and counterclockwise rotation of the main drive gear 220. In other embodiments, the valve drive gear 221 and the valve gear 222 are a unitary gear. As shown in the embodiment of FIG. 2a, the combination of the valve drive gear 221 and valve gear 222 may form an intermittent rack-and-pinion gear with a sliding valve.

The intermittent gear movement ensures that the valve 223 does not move continuously, but at discrete intervals coordinated with the timing of the assay steps. In the embodiment shown in FIG. 2a, the valve 223 takes the form of a linearly sliding elastomeric block with at least one fluid channel 233 extending therethrough at specific positions. In another embodiment shown in FIGS. 2e and 2f, the valve 223 takes the form of a rotating valve with at least one fluid channel 233 extending therethrough at specific rotational positions. Because specific positions and/or orientations of the valve 223 within the first cartridge mounting element 210 or wall 230 serve to complete and/or break fluid circuits within the cartridge system 200, position of the valve 223 determines what fluid in the assay flows where at a particular time point in the assay.

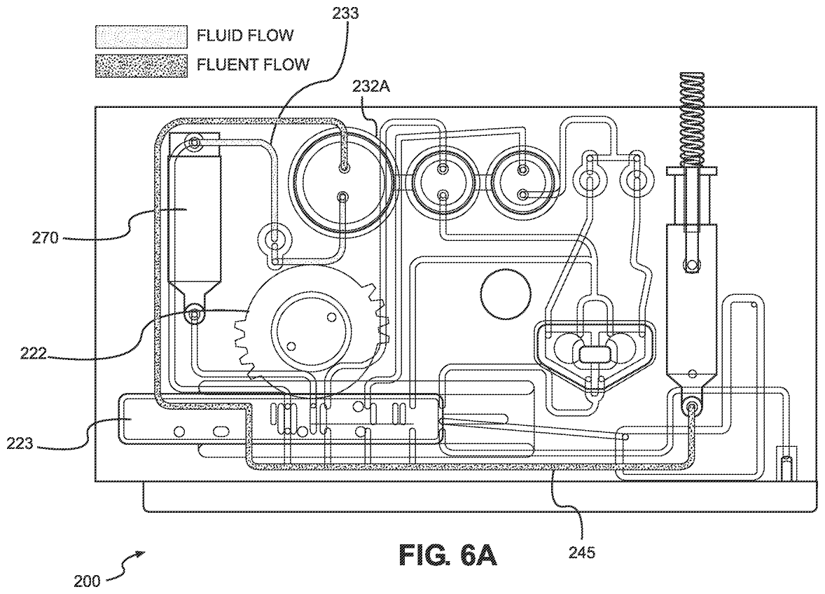

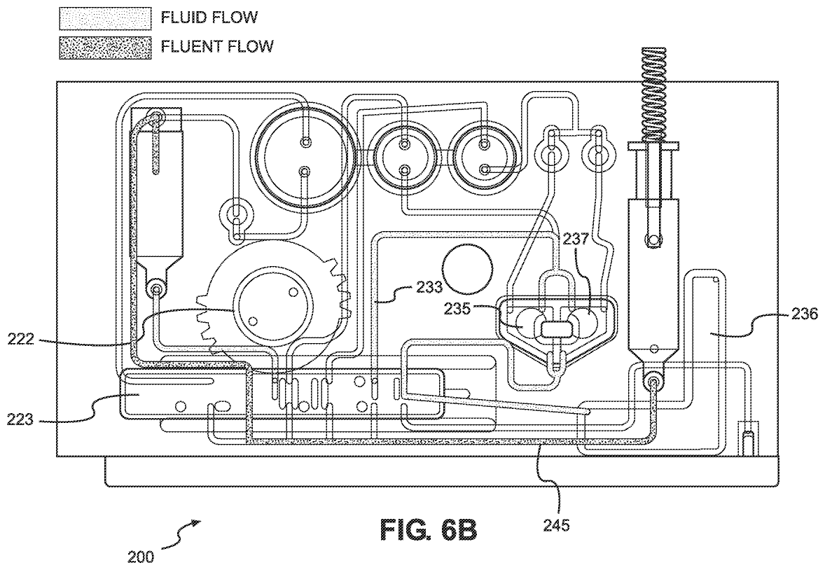

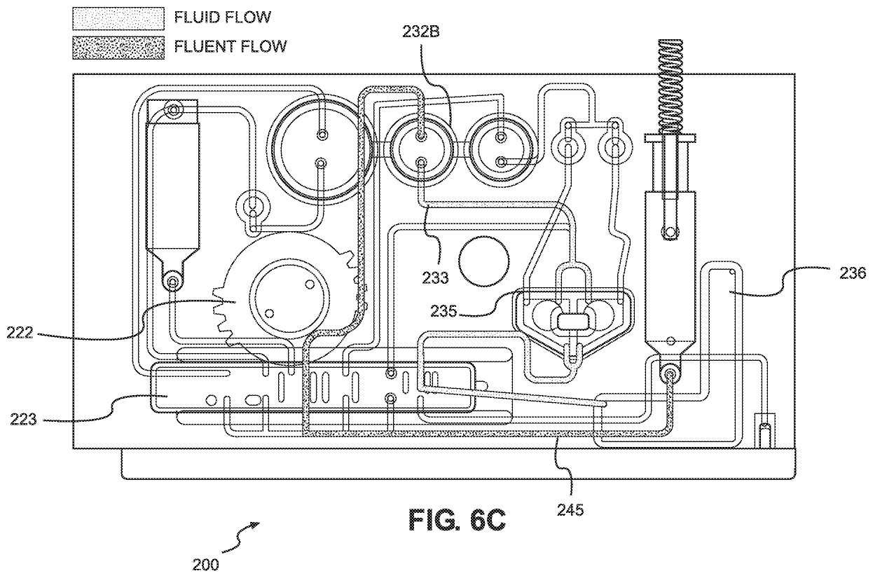

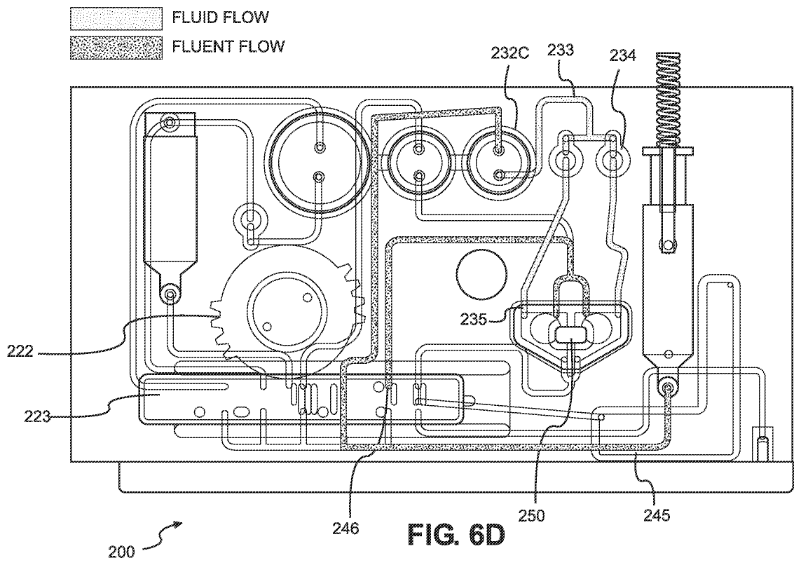

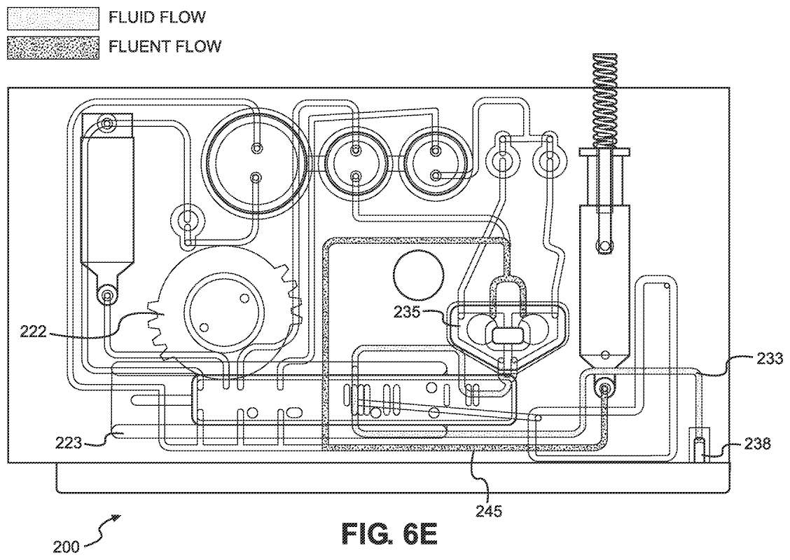

In certain embodiments, shown in operation in FIGS. 6a through 6e, a fluent channel 245 is adjustably connected with other portions of the cartridge system 200 based on the linear and/or rotational position of the valve 223. A pressurization mechanism is connected to the fluent channel 245 to provide a fluent flow 247 made up of fluent material to drive fluid flow. In certain embodiments, the fluent flow 247 can be provided by use of a fluent pump that is controlled by the system controller 212. In certain embodiments the fluent material is a gas, such as, but not limited to, air, nitrogen, an inert gas, or any combination thereof. In certain embodiments the fluent material is a liquid, such as, but not limited to, water, oil, or any combination thereof. In certain embodiments, the fluent flow 247 is isolated from ambient atmosphere to prevent contamination. In such embodiments, for example, the cartridge system 200 can form a closed system.

As shown in FIGS. 2a and 2b, the cam drive gear 224 is optionally interconnected with a cam 225 such that rotation of the cam drive gear 224 rotates the cam 225. The cam drive gear 224 may be a spur gear. A syringe housing 240 is fixedly connected to the first cartridge mounting element 210 or wall 230, with a syringe barrel 241 slidably located therein. This combination of the syringe housing 240 and syringe barrel 241 creates another embodiment of the pressurization mechanism. In this embodiment, the syringe housing may have a volume of 0.1 to 10 mL.

In the embodiment shown in FIGS. 2a through 2d, one segment of the syringe barrel 241 is connected to a syringe follower 242 sliding reciprocally in a syringe guide slot 243. Rotation of the cam 225 raises the syringe follower 242 to pull fluent material into the syringe housing 241. A syringe spring 244 located on an upper end of the syringe barrel 241 and connected to the second cartridge mounting element 230 biases the syringe barrel 241 in a downward direction once the rotation of cam 225 allows descent of the syringe follower 242. This expels the fluent material from the syringe housing 241 and into the fluent channel 245 adjustably connected with other portions of the cartridge system 200 to drive fluid flow.

In the embodiment shown in FIG. 2f, one end of the syringe barrel 241 is contacts a syringe follower 242. Rotation of the cam 225 can move the syringe follower 242 towards the syringe barrel 241 to expel fluent material from the syringe housing 241 into the fluent channel 245 adjustably connected with other portions of the cartridge system 200 to drive fluid flow. The syringe spring 244 biases the syringe barrel 241 in an outward direction once the rotation of cam 225 allows the syringe follower 242 to move away from the syringe barrel 241. This pulls the fluent material into the syringe housing 241.

As shown in FIGS. 2a through 2f, the first or second cartridge mounting element 210 or 230 include a sample receiver 231 to receive the sample system 100 and the sample contained therein. The sample receiver 231 may also include a sample heater 270 to heat the sample, by way of non-limiting example, to lyse and hybridize the sample. In certain embodiments, the sample heater 270 may vary the temperature of the sample from 20 degrees C. to 100 degrees C. In certain embodiments, the sample heater 270 may increase, decrease, and/or hold the sample temperature stable for any given time period according to any pattern required by a given reaction.

At least one reagent receiver 232 is configured to receive at least one reagent canister 260. Insertion of the reagent canisters 260 into their corresponding reagent receivers 232 completes a fluid circuit in the second cartridge mounting element 230 and allows the assay to proceed. Such an insertion may be the trigger to begin the assay, while other embodiments merely require insertion of the sample system 100 into the sample receiver 231. A plurality of fluid channels 233 extend between and interconnect the sample receiver 231, reagent receivers 232, a sample meter 234, a reaction chamber 235, a waste chamber 236, a collection chamber 238, and a vent port 246. In certain embodiments, at least one of the fluid channels 233 may serve as the reaction chamber 235. In certain embodiments, at least one of the fluid channels 233 may have a serpentine, spiral, or circular configuration to enable dynamic fluidic mixing.

The sample meter 234 can be used to ensure even combination of the reagents in the reaction chamber 235 and can also be used to provide a store of lyophilized reagents to combine with the assay fluid. While the sample meter 234 of the embodiment shown in FIG. 2a splits the fluid flow into two streams and utilizes two chambers, other embodiments may use more or fewer streams and chambers. The waste chamber 236 retains waste from the assay process. Such waste can be on the order of 1.5 mL to 2.0 mL. The collection port 238 is used to capture the products of the assay and may include some sort of sensing or testing mechanism. The vent port 246 is used to allow fluent material to escape from the cartridge system 300 and prevent overpressure.

As shown in FIGS. 2a through 2f, the reaction chamber 235 is used for processing the sample as required by the assay. Embodiments which use magnetic beads will include at least one magnet 237 surrounding at least part of the reaction chamber 235 in order to hold the beads in place during assays. The reaction chamber 235 also includes at least one mixing mechanism 250 for combining substances. In one embodiment, the mixing mechanism 250 is at least one vibrating actuator connected to at least one flexible membrane forming at least one wall of the reaction chamber 235. Vibration of the actuator causes oscillation of the membrane and generate mixing currents in the reaction chamber 235. The reaction chamber 235 may also include at least one reaction sensor 251 to detect the results of the assay process and communicate them to the readout 214. The reaction chamber 235 may also include at least one reaction heater 252 similar to the sample heater 270 for heat-treating the material within the reaction chamber 235. In certain embodiments, the reaction chamber 235 is located within the valve 223. The reaction chamber 235 may also include at least one porous substrate. In certain embodiments, the reaction chamber 235 is in the form of a tortuous, curved, or serpentine channel, which serves as an integral mixing mechanism. Alternative mixing mechanisms can include mechanical stir bars. Additional alternative mixing mechanisms can include multiple chambers that permit moving fluid back and forth (“shuttle mixing”) between the chambers. Any mixing mechanism described herein can additionally or alternatively be included in any part, portion, or element of the system, including any chamber described herein (e.g., reaction chamber 235, waste chamber 236, collection chamber 238) or any portion of any fluid channel 233 upstream or downstream of any chamber herein (e.g., reaction chamber 235, waste chamber 236, collection chamber 238), such as upstream of a reaction chamber 235 or downstream of a reaction chamber 235.

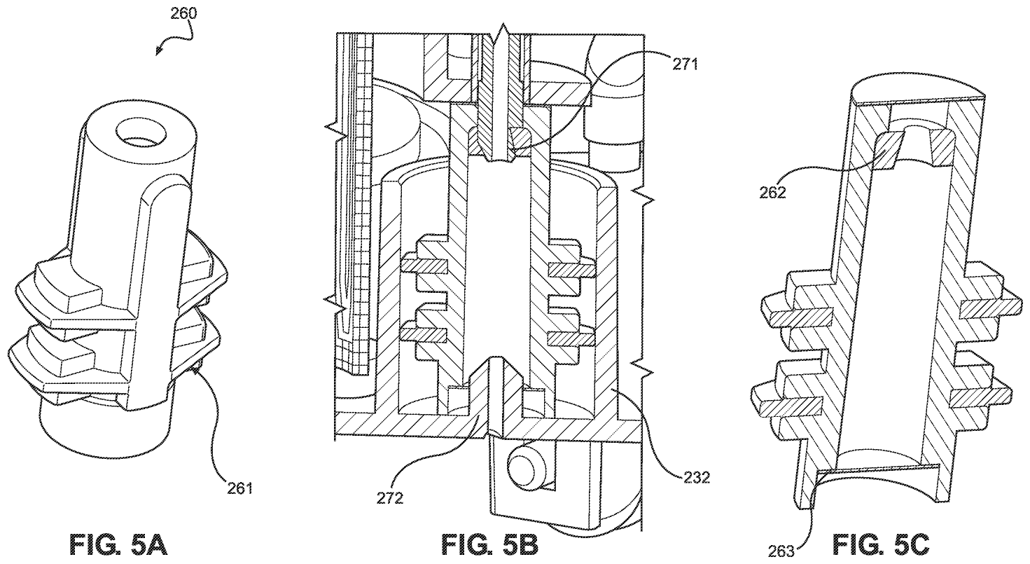

In certain embodiments, the reagent canisters 260 may have a structure similar to the sample receptacle 130, as shown in FIGS. 5a, 5b, and 5c. In this embodiment, the reagent canister includes guide projections 261 overmolded onto the outer surface of the reagents canister 260 to guide insertion of the reagent canister 260 into the cartridge system 200. The reagent canisters 260 may contain a fluid or lyophilized consumable, such as, but not limited to, a prepared reagent or reagents for combination with the sample. The reagent canister 260 provides for reagent stability during storage via hermetic sealing means such as foil laminate packaging over a reagent aperture 262 and/or a delivery aperture 263. A pneumatic spike 271 may be inserted through the reagent aperture 261 to pressurize the reagent canister 260. Upon insertion, the pneumatic spike 271 punctures or cuts open the foil laminate packaging and forms a pneumatic seal with the reagent aperture 262. Once the reagent and sample are combined, a fluidic spike 272 may open the reagent canister 260 opposite the pneumatic spike 271, extending through the delivery aperture 263 to allow egress to the cartridge system 200.

The following is an exemplary use of the sample system 100 and cartridge system 200 to capture, amplify, and detect one or more nucleic acids from any nucleic acid containing target in a variety of clinical samples. This use is an illustrative example only, and should not be taken to imply any limitation on the use of the sample system 100 and/or the cartridge system 200. While this cartridge system 200 includes first, second, and third reagent receivers 232a, 232b, and 232c due to the process used, other cartridge systems 200 for use in different procedures may have different numbers of reagent receivers 232. The fluid circuits for the varying steps in the assay process are shown in FIGS. 6a through 6e.

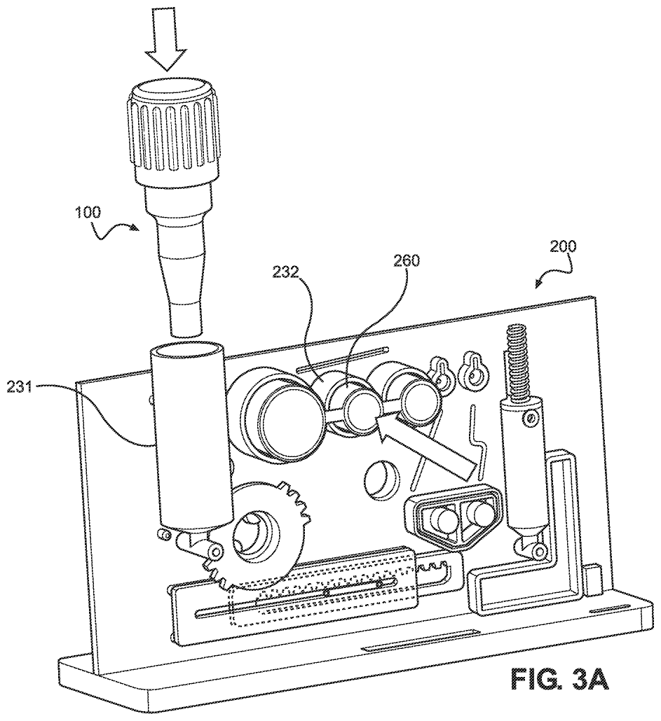

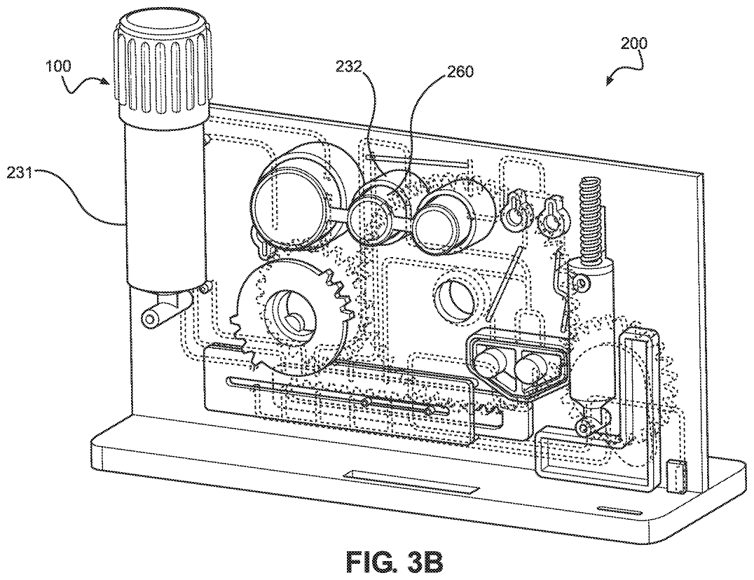

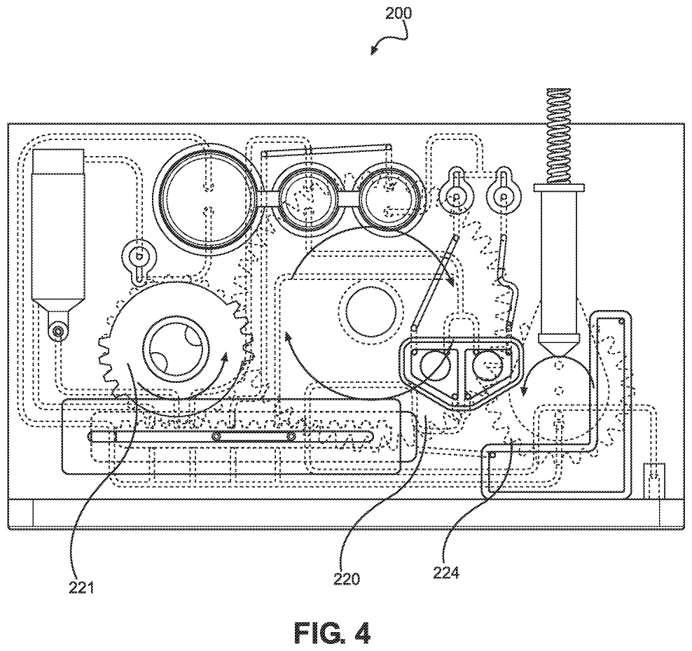

A biological sample can be obtained with the sample system 100 by collecting a sample on the sample holder 110. As seen in FIG. 3a, the sample system 100 can then be placed in the sample receiver 231 of the cartridge system 200.

As seen in FIG. 6a, during use, the first fluid movement is adding a buffer (referred to elsewhere herein as a “first buffer”) to the sample receptacle 130 (not shown). By driving the valve gear 222, the valve 223 is positioned to create an open fluid circuit between the fluent channel 245 and a first reagent receiver 232a. By driving the cam drive gear 224 (not shown), fluent material will be driven through the connecting fluid channel 233 and the valve 223. The buffer will pass from the first reagent receiver 232a to the sample receptacle 130, submerging the sample holder 110 (not shown) in the buffer. In some versions, the buffer from the first reagent receiver 232a contains a number of reagents, such as one or more lysis reagents, one or more hybridization reagents (e.g., capture oligonucleotides configured to bind both to a nucleic acid target and a solid substrate), and/or one or more solid substrates (e.g., beads, such as magnetic beads). In some versions, one or more of the reagents, such as the solid substrate, is present in a fluid channel 233 between the first reagent receiver 232a and the sample receptacle 130, thereby reconstituting such reagent(s) and delivering them to the sample receptacle 130. In yet other versions, one or more of the reagents may be stored in the sample reservoir 121 and thereby delivered to the sample receptacle 130 and sample holder 110 during the sample collection such that delivery of the reagents from a reagent receiver 232 is not required.

Once the sample holder 110 is fully submerged and inserted into the cartridge system 200, the system may enter into a heating cycle using the sample heater 270 to lyse cells and/or viruses in the sample to release target nucleic acids. Upon cooling, the released target nucleic acids can hybridize to the solid substrate either directly or via capture oligonucleotides to generate target nucleic acid complexes. Other chemistries, such as silica solid phase extraction, which may or may not require heat for lysis and would bind non-specifically (i.e. all nucleic acids) to a solid support (typically magnetic beads but also filters or frits etc.) can also or alternatively be used.

As seen in FIG. 6b, once the target nucleic acid complexes are generated, the fluid is transferred to the reaction chamber 235 where the target nucleic acid complexes are immobilized therein and the remaining fluid is flowed to the waste chamber 236. By driving the valve gear 222, the valve 223 is positioned to create an open fluid circuit between the fluent channel 245 and the sample receptacle 130 (not shown), the sample receptacle 130 and the reaction chamber 235, and the reaction chamber 235 and the waste chamber 236. In the exemplary version, the solid substrate is a magnetic bead, and the target nucleic acid complexes are immobilized in the reaction chamber 235 with a magnet 237 while the fluid is passed to the waste chamber 236. In other versions, the reaction chamber 235 contains a porous substrate, such as a frit, filter, or other material capable of capturing target nucleic acid complexes in the reaction chamber 235 via size exclusion or inclusion while the fluid passes through. Other mechanisms for capturing the target nucleic acid complexes in the reaction chamber 235 while separating the fluid therefrom can be used.

As seen in FIG. 6c, following the target nucleic acid complexes being collected in the reaction chamber 235, they can optionally be washed by flowing a wash solution over them. By driving the valve gear 222, the valve 223 is positioned to create an open fluid circuit between the fluent channel 245 and a second reagent receiver 232b, the second reagent receiver 232b and the reaction chamber 235, and the reaction chamber 235 and the waste chamber 236. The wash solution can then be transferred from the second reagent receiver 232b through the reaction chamber 235, thereby washing the immobilized target nucleic acid complexes, and to the waste chamber 236. In some versions, one wash is performed. In some versions, multiple washes are performed. The multiple washes can be performed with wash solution from the second reagent receiver 232b or with wash solutions from different reagent receivers 232.

As seen in FIG. 6d, following the target nucleic acid complexes being collected and washed, the system 200 will fill the reaction chamber 235 with a reaction mix from a third reagent receiver 232c for amplification of the target nucleic acid. By driving the valve gear 222, the valve 223 is positioned to create an open fluid circuit between the fluent channel 245 and the third reagent receiver 232c, the third reagent receiver 232c and the sample meter 234, the sample meter 234 and the reaction chamber 235, and the reaction chamber 235 and the vent port 246. The reaction mix can then be transferred from the third reagent receiver 232c into the reaction chamber 235. The reaction mix can contain amplification reagents required for amplification, such as nucleic acid polymerases, primers, dNTPs, salts, etc. The mixing mechanism 250 will ensure the amplification reagents are in a fully homogenous solution. Once the reaction mix is transferred to the reaction chamber 235, application can be performed. In some versions, the amplification is performed with the target nucleic acids still immobilized within the reaction chamber 235 in the form of the target nucleic acid complexes. In some versions, the target nucleic acids are first eluted from the immobilized solid substrate before amplification is performed. The elution can occur by flowing an elution buffer from a fourth reagent receiver 232 (not shown in the exemplary versions) into the reaction chamber 235. In some versions, the eluted target nucleic acids are transferred to a different reaction chamber 235 before amplification is performed.

As seen in FIG. 6e, once the amplification process is complete, the contents of the reaction chamber 235 will be driven to the collection chamber 238 or the readout 214 (not shown). By driving the valve gear 222, the valve 223 is positioned to create an open fluid circuit between the fluent channel 245 and the reaction chamber 235, and the reaction chamber 235 and the collection chamber 238 or the readout 214.

Once the process is complete, the valve 223 is positioned to close, ending in a final sealed position isolating the collection chamber 238 or the readout 214 from the remainder of the cartridge system 200.

The readout 214 can comprise any device or system capable of detecting amplified nucleic acid and displaying the detection to a user. A number of such devices or systems are known in the art. Exemplary readouts 214 include visual flow strips, fluorescent detection systems, etc.

The target nucleic acid can be comprised by or derived from a sample. The sample can comprise a sample obtained or derived from a subject (i.e., a clinical sample), a synthetic sample, or any other type of sample potentially containing a target nucleic acid. Examples of clinical samples include whole blood, serum, plasma, sputum, saliva, nasopharyngeal swab, stool, anal swab, vaginal swab, urine, dry blood spot, penile swab, urethral swab, and skin swab. “Whole blood” as used herein refers to blood drawn from the body from which none of the components, such as plasma or platelets, has been removed. Whole blood can comprise components in addition to those originally present, such as EDTA and/or other components.

The target nucleic acid can include any type of nucleic acid. The target nucleic acid can comprise DNA or RNA. The nucleic acid can be single stranded or double stranded. Exemplary types of DNA include genomic DNA, cDNA, and extrachromosomal DNA, among others. Exemplary types of RNA include mRNA, tRNA, rRNA, and μRNA, among others. The target nucleic acid can comprise a sequence of interest. The sequence of interest, for example, can be a sequence indicative of, or unique to, a particular cell, pathogen, bacterium, virus, disease state, mutation status, genetic characteristic, or other item of interest.

Exemplary lysis reagents can include detergents. Exemplary detergents include anionic detergents, cationic detergents, nonionic detergents, and zwitterionic detergents. Anionic detergents are preferred. Exemplary anionic detergents include soaps, alkylbenzene sulfonates, alkyl sulfonates, alkyl sulfonates, alkyl sulfates, salts of fluorinated fatty acids, silicones, fatty alcohol sulfates, polyoxyethylene fatty alcohol ether sulfates, α-olefin sulfonate, polyoxyethylene fatty alcohol phosphates ether, alkyl alcohol amide, alkyl sulfonic acid acetamide, alkyl succinate sulfonate salts, amino alcohol alkylbenzene sulfonates, naphthenates, alkylphenol sulfonate and polyoxyethylene monolaurate. Specific exemplary anionic detergents include sodium octyl sulfate, potassium oleate, sodium dodecyl sulfate, lithium dodecyl sulfate, butylnaphthalenesulfonic acid sodium salt, sodium decyl sulfate, sodium 1-butanesulfonate, sodium dodecylbenzenesulphonate, sodium stearate, magnesium stearate, 1-dodecanesulfonic acid sodium salt, sodium allylsulfonate, dodecylbenzenesulfonic acid sodium salt, calcium dodecylbenzene sulfonate, ammonium lauryl sulfate, and sodium lauryl polyoxyethylene ether sulfate, among others. Preferred detergents include dodecyl sulfate salts such as sodium dodecyl sulfate (SDS) or lithium dodecyl sulfate, or others. Other preferred detergents include lauroyl sarcosinate salts, such as sarkosyl (sodium lauroyl sarcosinate). Other exemplary lysis reagents can include chaotropes, such as guanidinium thiocyanate and guanidinium hydrochloride.

A protease can be included in the first buffer. The protease can be included to digest proteins such as nucleases or other proteins present in the sample. The proteins may be released into solution after cell lysis. The digestion of proteins such as nucleases can protect the nucleic acids in the sample from nuclease attack. Any protease or combination of proteases can be included. An exemplary protease is proteinase K, which is a broad-spectrum protease.

The solid substrate can comprise a bead, a membrane, or any other type of solid substrate. The solid substrate should be capable of maintaining the target nucleic acid in a solid phase when in contact with the liquid phase of the first buffer and when the liquid phase of the first buffer is removed from the solid phase. Exemplary beads include magnetic beads (e.g., Dynabeads® (ThermoFisher Scientific), polymeric beads (e.g., polystyrene), glass beads, etc. Exemplary membranes include polymer membranes (e.g., polyethersulfone, nylon, polytetrafluoroethylene, polycarbonate, nitrocellulose), glass fiber membranes, cellulose membranes, and highly matrixed membranes.

The hybridization reagents can include capture oligonucleotides. The capture oligonucleotides can comprise a sequence capable of hybridizing to specific sequences of target nucleic acids and another moiety capable of connecting either directly or indirectly to the solid substrate. Various mechanisms for connecting a capture oligonucleotide to a solid substrate are known, and include streptavidin and biotin pairs, hybridizable nucleic acid sequences (e.g., poly(A) and poly(T) sequences), antibody and antigen pairs, G-quadruplex structure and G-quadruplex-binding protein pairs; aptamer and aptamer target pairs, and ion/anion binding pairs.

The porous substrate can separate the first buffer from the solid substrate via size exclusion and thereby capture the solid substrate on or in the porous substrate. The solid substrate in such versions can comprise a bead, a filament, etc. “Porous substrate” refers to any porous solid or semi-solid substrate that permits a fluid such as a liquid to flow therethrough. The porous substrate may be configured to permit certain solids or particles having a certain size or physicochemical characteristic to flow therethrough, while capturing or filtering others. Examples include polymeric, ceramic, or other types of filters, frits, or membranes.

As used herein, “nucleic acid amplification” encompasses reverse transcription of RNA to DNA, copying of DNA, and combinations thereof. The nucleic acid amplification can comprise any method suitable for amplifying nucleic acids. Exemplary methods comprise thermocycling amplification, such as the polymerase chain reaction (PCR), and isothermal amplification. A number of isothermal amplification methods are known in the art. These include transcription mediated amplification (TMA), nucleic acid sequence-based amplification (NASBA), signal mediated amplification of RNA technology (SMART), strand displacement amplification (SDA), nicking enzyme amplification reaction (NEAR), rolling circle amplification (RCA), loop-mediated isothermal amplification of DNA (LAMP), isothermal multiple displacement amplification (MDA), helicase-dependent amplification (HDA), single primer isothermal amplification (SPIA), and cross primed amplification (CPA). See, e.g., Notomi et al. 2000 (Notomi T, Okayama H, Masubuchi H, Yonekawa T, Watanabe K, Amino N, Hase T. Loop-mediated isothermal amplification of DNA. Nucleic Acids Res. 2000 Jun. 15; 28(12):E63), U.S. Pat. Nos. 6,410,278; 6,743,605; 6,764,821; 7,494,790; 7,468,245; 7,485,417; 7,713,691; 8,133,989; 8,206,902. U.S. Pat. Nos. 8,288,092; 8,445,664; 8,486,633; and 8,906,621. Software and other methods for designing primers suitable for use in such isothermal amplification methods are well-known in the art. See, e.g., PrimerExplorer LAMP primer designing software from Eiken Chemical, Kimura et al. 2011 (Kimura Y, de Hoon M J, Aoki S, Ishizu Y, Kawai Y, Kogo Y, Daub C O, Lezhava A, Amer E, Hayashizaki Y. Optimization of turn-back primers in isothermal amplification. Nucleic Acids Res. 2011 May; 39(9):e59), and others.

In the embodiments shown in FIGS. 7a through 7f, the cartridge frame 205 includes a first cartridge mounting element 210 and a separable second cartridge mounting element 230. This permits certain elements of the cartridge system 200, such as those mounted to the first cartridge mounting element 210, to be reused after use. Exemplary elements that can be mounted to the reusable first cartridge mounting element 210 can include any one or more of the power source 211, the system controller 212, the gear actuator 215, the main drive gear 220, the valve drive gear 221, the cam drive gear 224, the cam 225, and the sample heater(s) 270, in any combination, as shown in FIGS. 7b and 7c. Alternate embodiments may include additional reusable elements, such as elements of the gear train and pressurization mechanism, and the readout 214. Non-reusable elements can be mounted to the disposable second cartridge mounting element 230. Exemplary elements that can be mounted to the second cartridge mounting element 230 can include any one or more of the valve gear 222, the valve 223, the sample receiver 231, reagent receivers 232, fluid channels 233, the reaction chamber 235, syringe housing 240, syringe barrel 241, syringe follower 242, syringe spring 244, and fluent channel 245, in any combination, as shown in FIGS. 7d through 7f. After conducting an assay, a user may remove the used second cartridge mounting element 230 and insert a new second cartridge mounting element 230 into the first cartridge mounting element 210 to conduct another assay.

Any amounts disclosed herein are provided on a weight basis unless explicitly indicated otherwise or the context clearly indicates otherwise. For example, amounts provided as % are % w/w. Amounts provided as ratios are ratios by weight. The amounts of each of the components in the composition may be varied from the amounts described herein depending upon the nature of the component, the weight and volume of the material to be treated, and the effects desired. Those of ordinary skill in the art will be able to adjust component amounts as required.

Any version of any component or method step of the invention may be used with any other component or method step of the invention. The elements described herein can be used in any combination whether explicitly described or not. All combinations of method steps as used herein can be performed in any order, unless otherwise specified or clearly implied to the contrary by the context in which the referenced combination is made.

As used herein, the singular forms “a,” “an,” and “the” include plural referents unless the content clearly dictates otherwise. Numerical ranges as used herein are intended to include every number and subset of numbers contained within that range, whether specifically disclosed or not. Further, these numerical ranges should be construed as providing support for a claim directed to any number or subset of numbers in that range. For example, a disclosure of from 1 to 10 should be construed as supporting a range of from 2 to 8, from 3 to 7, from 5 to 6, from 1 to 9, from 3.6 to 4.6, from 3.5 to 9.9, and so forth.

Any and all patents, patent publications, and peer-reviewed publications (i.e., “references”) cited herein are expressly incorporated by reference in their entirety to the same extent as if each individual reference were specifically and individually indicated as being incorporated by reference. In case of conflict between the present disclosure and the incorporated references, the present disclosure controls.

The devices, methods, compounds and compositions of the present invention can comprise, consist of, or consist essentially of the essential elements and limitations described herein, as well as any additional or optional steps, ingredients, components, or limitations described herein or otherwise useful in the art.

While this invention may be embodied in many forms, what is described in detail herein is a specific embodiment of the invention. The present disclosure is an exemplification of the principles of the invention is not intended to limit the invention to the particular embodiments illustrated. It is to be understood that this invention is not limited to the particular examples, method steps, and materials disclosed herein as such method steps and materials may vary somewhat. It is also understood that the terminology used herein is used for the purpose of describing particular embodiments only and is not intended to be limiting.

1. A system for assay of a biological sample, the system comprising a cartridge system, the cartridge system comprising:
a cartridge frame to which is mounted a sample receiver connected to or configured to connect to a sample receptacle, at least one reagent receiver, at least one valve, and at least one reaction chamber;
a plurality of fluid channels extending between the at least one valve, the sample receiver, the at least one reagent receiver, and the at least one reaction chamber;
at least one fluent channel extending from a pressurization mechanism to the at least one valve; and
a main drive gear, wherein rotation of the main drive gear determines the position of the at least one valve,
wherein the position of the at least one valve determines which of the plurality of fluid channels is operably connected to the fluent channel.

2. The system of claim 1, wherein the sample receiver comprises a sample heater operably connected to a system controller.

3. The system of claim 1, wherein the pressurization mechanism comprises a fixedly mounted syringe housing, and a syringe barrel slidably disposed within the syringe housing, wherein the fluent channel extends from the syringe housing to the at least one valve.

4. The system of claim 1, wherein the main drive gear is connected to a cam drive gear driving a rotating cam.

5. The system of claim 4, wherein the syringe barrel is connected to a syringe follower, the syringe follower being in contact with the cam.

6. The system of claim 4, wherein the syringe barrel is connected to a syringe spring, the syringe spring biasing the syringe barrel relative to the syringe housing.

7. The system of claim 4, wherein the main drive gear is a worm gear, and the cam drive gear comprises at least one cam gear ratchet and at least one cam gear pawl, wherein the combination of the at least one cam gear ratchet and at least one cam gear pawl forms an intermittent gear by allowing rotation of the cam drive gear in only one direction.

8. The system of claim 1, wherein:
the cartridge frame comprises a power source operably connected to a system controller;
the system controller comprises a timer;
the system controller comprises a gear actuator controlled by the timer, the gear actuator driving the main drive gear;
the cartridge frame further comprises a readout;
the at least one reaction chamber comprises a mixing mechanism operably connected to the system controller;
the mixing mechanism comprises at least one vibrating actuator connected to at least one flexible membrane, wherein the at least one flexible membrane forms at least one wall of the at least one reaction chamber;
the at least one reaction chamber comprises a sample heater operably connected to the system controller;
the at least one reaction chamber comprises a magnet.

9-15. (canceled)

16. The system of claim 1, wherein the at least one reaction chamber is located within the at least one valve.

17. The system of claim 1, wherein the main drive gear is connected to at least one valve drive gear driving the at least one valve.

18. The system of claim 17, wherein a combination of the at least one valve drive gear and at least one valve gear forms an intermittent gear through incomplete toothing of the at least one valve drive gear.

19. The system of claim 18, wherein the intermittent gear is an intermittent rack-and-pinion gear, and the at least one valve is a sliding valve.

20. The system of claim 17, wherein the main drive gear is a worm gear, and the at least one valve drive gear comprises at least one valve gear ratchet and at least one valve gear pawl, wherein the combination of the at least one valve gear ratchet and at least one valve gear pawl forms an intermittent gear by allowing rotation of the at least one valve drive gear in only one direction.

21. The system of claim 20, wherein the at least one valve is a rotating valve.

22. The system of claim 1, wherein the cartridge frame comprises a first cartridge mounting element and a second cartridge mounting element separable from the first cartridge mounting element, the first cartridge mounting element includes the main drive gear mounted thereto, and the second cartridge mounting element comprises the sample receiver, the at least one reagent receiver, and the at least one valve mounted thereto.

23. The system of claim 1, further comprising a sample system, the sample system comprising:
the sample receptacle receiving the biological sample; and
a sample cap, wherein a sample reservoir is located within the sample cap,
wherein:
a sample holder is connected to the sample cap;
the sample receptacle further comprises at least one reservoir piercer; and
the at least one reservoir piercer is placed within the sample receptacle such that the sample reservoir is a pierced by the at least one reservoir piercer when the sample cap is connected to the sample receptacle.

24-26. (canceled)

27. The system of claim 1:
wherein the sample receiver comprises a sample heater operably connected to a system controller;
wherein the pressurization mechanism comprises a fixedly mounted syringe housing, and a syringe barrel slidably disposed within the syringe housing, wherein the fluent channel extends from the syringe housing to the at least one valve;
wherein the main drive gear is connected to a cam drive gear driving a rotating cam;
wherein the syringe barrel is connected to a syringe follower, the syringe follower being in contact with the cam;
wherein the syringe barrel is connected to a syringe spring, the syringe spring biasing the syringe barrel relative to the syringe housing;
wherein the system controller comprises a timer;
wherein the system controller comprises a gear actuator controlled by the timer, the gear actuator driving the main drive gear;
wherein the cartridge frame further comprises a readout;
wherein the at least one reaction chamber comprises a mixing mechanism operably connected to the system controller;
wherein the at least one reaction chamber comprises a sample heater operably connected to the system controller;
wherein the main drive gear is connected to at least one valve drive gear driving the at least one valve;
wherein a combination of the at least one valve drive gear and at least one valve gear forms an intermittent gear through incomplete toothing of the at least one valve drive gear, wherein the intermittent gear is an intermittent rack-and-pinion gear, and the at least one valve is a sliding valve, or wherein the main drive gear is a worm gear, the cam drive gear comprises at least one cam gear ratchet and at least one cam gear pawl, the combination of the at least one cam gear ratchet and at least one cam gear pawl forms an intermittent gear by allowing rotation of the cam drive gear in only one direction, the at least one valve drive gear comprises at least one valve gear ratchet and at least one valve gear pawl, the combination of the at least one valve gear ratchet and at least one valve gear pawl forms an intermittent gear by allowing rotation of the at least one valve drive gear in only one direction, and the at least one valve is a rotating valve.

28. A method of using the system of claim 1, the method comprising:
complexing a target nucleic acid in a first buffer in the sample receptacle with a solid substrate to generate a target nucleic acid complex in the first buffer;
transferring the first buffer comprising the target nucleic acid complex from the sample receptacle to the at least one reaction chamber;
immobilizing the target nucleic acid complex in the at least one reaction chamber;
removing the first buffer from the at least one reaction chamber and the target nucleic acid complex immobilized therein;
transferring an amplification reaction mix to the at least one reaction chamber; and
amplifying the target nucleic acid in the amplification reaction mix in the at least one reaction chamber to generate amplified nucleic acid.

29. The method of claim 28, wherein:
the method further comprises transferring the first buffer from a first reagent receiver to the sample receptacle;
the first buffer, prior to complexing with the target nucleic acid, comprises the solid substrate;
the first buffer, prior to complexing with the target nucleic acid, comprises a lysis reagent;
the method further comprises heating the first buffer and the target nucleic acid in the sample receptacle;
the immobilizing the target nucleic acid complex in the at least one reaction chamber comprises immobilizing the target nucleic acid complex in the at least one reaction chamber with a magnet or immobilizing the target nucleic acid complex in a porous substrate contained within the at least one reaction chamber;
the method further comprises, before removing the first buffer from the at least one reaction chamber and prior to transferring the amplification reaction mix to the at least one reaction chamber, washing the immobilized target nucleic complex in the at least one reaction chamber wherein the washing comprises transferring a wash solution from a second reagent receiver to and through the at least one reaction chamber;
the transferring the amplification reaction mix to the at least one reaction chamber comprises transferring the amplification reaction mix from a third reagent receiver to the at least one reaction chamber; and
the method further comprises, after the amplifying the target nucleic acid, transferring the reaction mix comprising the amplified nucleic acid to a readout, wherein the amplified nucleic acid is detected with the readout.

30-36. (canceled)
